(19) United States
(12) Patent Application Publication
(10) Pub. No.: US 2003/0129180 A1
(43) Pub. Date: Jul. 10, 2003

(54) Products and methods for treating microbial infections

(75) Inventors: Keith Homer Baker, West Chester Township, OH (US); Rowan Andrew Grayling, Loveland, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Proctor & Gamble Company

(21) Appl. No.: 10/209,203

(22) Filed: Jul. 31, 2002

Related U.S. Application Data
(60) Provisional application No. 60/309,329, filed on Aug. 1, 2001.

(51) Int. Cl. C12N 9/24; A61K 38/47

(52) U.S. Cl. 424/94.61; 435/200

(57) ABSTRACT

A method for treating athlete's foot involves treating both the infected skin and the shoes of a subject suffering from athlete's foot. Skin treatment compositions and shoe treatment compositions can be used concurrently to treat the infection.

### CROSS REFERENCE TO RELATED APPLICATION

This application claims the benefit of U.S. Provisional Application No. 60/309,329, filed Aug. 1, 2001.

### FIELD OF THE INVENTION

This invention relates to products, including compositions, and methods for treating microbial, particularly fungal, infections. The products and methods can be used to treat fungal infections in shoes or on skin, or both. Compositions are provided that are particularly well suited for treating fungi that can cause athlete's foot in leather-containing shoes, such as athletic shoes, and methods and articles of manufacture employing same to treat the shoes prior to and/or during and/or after washing the shoes. Compositions are further provided that are particularly well suited for treating athlete's foot infections on skin, particularly of the foot.

### BACKGROUND

Normal human skin is colonized by large numbers of a variety of microorganisms, most of which live harmlessly on its surface as commensals, and are likely important for normal skin function. Species present include both gram-positive and gram-negative bacteria, as well as lesser populations of fungi that include yeasts and sometimes dermatophytic (skin-infecting) fungi (Aly, R., 1994, J. Am. Acad. Dermatol. 31(3):S21-25; Laboratory Handbook of Dermatophytes, 1997, J. Kane, ed., Star Publishing Company, Belmont, Calif.). Occasionally, both these resident flora as well as transient flora acquired from the environment (species that are found temporarily on the skin) can initiate or potentiate persistent skin and nail infections.

Fungal infections of the skin and nails are in fact among the most common skin disorders worldwide. Such infections are most often initiated by dermatophytic fungi, and can develop into serious diseases involving a complex interplay between both dermatophytes and overgrowth of the resident and transient bacterial species. Tinea pedis, commonly known as athlete's foot, is the most common form of skin disorder caused by dermatophytes, and most often occurs in the skin webbing between toes and on the soles of the feet (Leyden, J. L., 1994, J. Am. Acad. Dermatol. 31(3):S31-33; Laboratory Handbook of Dermatophytes, 1997, J. Kane, ed., Star Publishing Company, Belmont, Calif.). The majority of athlete's foot cases are caused by Trichophyton rubrum, Trichophyton mentagrophytes, and Epidermophyton floccosum, either separately or in combination. These fungi are transmitted by direct or indirect contact with skin flakes, hair, clothing, towels, and communal surfaces such as sports club locker room floors, carpets, and swimming pools. Depending on the species, such fungi can persist for long periods in these environments (Aly, R., 1994, J. Am. Acad. Dermatol. 31(3):S21-25).

In recent years the incidence of dermatophyte skin infections, particularly Tinea pedis, has been increasing steadily, particularly in developed countries, where as much as 10% of the population is affected at any one time (Laboratory Handbook of Dermatophytes, 1997, J. Kane, ed., Star Publishing Company, Belmont, Calif.). The increasing incidence is attributed predominantly to lifestyle changes that include an increased use of communal facilities such as sports clubs, as well as increased use of athletic shoes and closed footwear in general. In fact, the key factor identified in potentiating dermatophyte infections that cause tinea pedis is occlusion, where shoes provide an enclosed environment of high humidity and warmth that is ideal for dermatophyte growth (Aly, R., 1994, J. Am. Acad. Dermatol. 31(3):S21-25; Laboratory Handbook of Dermatophytes, 1997, J. Kane, ed., Star Publishing Company, Belmont, Calif.).

Products, compositions, and methods have been previously described that treat dermatophyte infections of the skin, provide antimicrobial coatings in shoes or socks, or attempt to sanitize shoes. For instance, the treatment of tinea pedis is accomplished by a number of over the counter (OTC) remedies whose actives include Miconazole nitrate, Terbinafine hydrochloride. Clotrimazole, Tolnaftate, and undecelynic acid. These products are marketed under such brand names as Dr. Scholl's and Desenex.

Similarly, multiple products are marketed to control foot and shoe odor. Examples of these would include “Desenex Sneaker Spray”, Dr. Scholl's Foot and Sneaker Spray” and Second Wind's “Deodorant and Disinfectant Spray”. Actives in the Second Wind system (EPA registration number 334-214-65192 &334-IL-2) include ethyl alcohol, 4-tert-amylphenol, and ortho-phenylphenol.

However, existing products can suffer from drawbacks such as having limited efficacy, disrupting the balance of the important resident skin flora, being expensive, being difficult or inconvenient to use, or destroying shoe components such as leather.

The use of fungal cell wall degrading enzymes for the biological control of fungi is described in the literature for a limited number of applications. Methods for using such enzymes have been disclosed for the control of plant fungal diseases, for food and cosmetic preservation uses, and for treating systemic and topical fungal infections in animals and humans. Methods have also been disclosed that describe the use of combinations of enzymes and traditional antimicrobial agents, such as chemicals and lytic peptides, to increase the effectiveness of enzyme-based antifungal treatments. Enzymes have certain advantages over traditional chemical antifungal agents, that include specificity of action, lack of toxicity, biodegradability, ease of engineering through modem molecular methods, and a low potential for generating resistance.

However, no methods have been found that use certain enzymes for specific treatment of dermatophytic fungi. Furthermore, no methods have been found for the concurrent treatment of shoes and feet.

### SUMMARY

This invention relates to products and methods for treating microbial infections. In one embodiment of the invention, the method comprises:

a) treating skin with a first organism-specific enzyme, or

b) treating a substrate that, directly or indirectly, covers the skin with a second organism-specific enzyme, or

c) treating the skin with the first organism-specific enzyme and treating the substrate with the second organism-specific enzyme. The first and second organism-specific enzymes may be the same or different.

The first organism-specific enzyme can be formulated in a topical composition and the second organism-specific enzyme can be formulated in a treating composition. For treating certain types of microbial infections, for example fungal infections, such as tinea pedis, the first and second organism-specific enzymes may each be independently selected from the group consisting of a chitinase, a glucanase, and combinations thereof. In this embodiment, the treating composition can be a shoe treating composition.

### DETAILED DESCRIPTION OF THE PREFERRED EMBODIMENTS

All U.S. patents cited herein are hereby incorporated by reference. All amounts, ratios, and percentages are by weight, unless otherwise indicated.

Definitions and Usage of Terms

“A” or “an” means one or more.

“Antifungal agent” means an ingredient used to treat fungal infections, not including the organism-specific enzyme of this invention.

“Organism-specific enzyme” means an enzyme, alone or in combination, that kills or weakens a microbial organism.

Dematophyte Infections

The care of shoes and feet remains an unsatisfactorily met need. Dermatophyte infections are often difficult to treat, having high relapse rates. Dermatophytes are readily isolated from the insides of shoes, shoe insoles, and from feet enclosed in shoes. Since shoes serve as an ideal environment for fungal growth, it is likely that they serve to both amplify the fungi present or inoculated from infected feet, and provide a source of fungi for continual reinfection of the feet (Jamieson, R. C., 1941, Arch. Derm. Syph. 44:837-844; Baer, R. L. et al., 1955, J. Invest. Dermatol. 24:619-662; Anonymous, 1962, Br. Med. J. 2:35-36). Without wishing to be bound by theory, it is thought that a cycle probably exists, whereby the foot inoculates the shoe, and the shoe re-inoculates the foot, with the shoe becoming the main reservoir for reinfection. Hence, treatment of dermatophyte skin infections in the absence of simultaneous, effective shoe treatments probably explains in part the high relapse rates observed. Concurrent elimination or reduction of dermatophytes on both the insides of shoes and the skin of the feet is thought to be an improved option to prevent and treat both primary dermatophyte infection and reinfection that cause athlete's foot. However, removing the reservoir by effective shoe treatment alone is also thought to be a good option to minimize the potential for reinfection.

Therefore, it is an object of this invention to provide more satisfactory anti-microbial treatments particularly anti-fungal treatments, for shoes. It is a further object of this invention to provide more effective treatments for fungi causing or associated with tinea pedis. It is a further object of this invention to combine the shoe treatments with shoe cleaning systems. It is a further object of this invention to combine the shoe treatments with treatments of the skin of the foot. It is a further object of the invention to provide systems to give the above benefits either separately or in combination with each other.

This invention reduces or eliminates dermatophytes present on the insides of shoes (including insoles), and secondarily on the skin of the feet. This invention effectively decreases the likelihood of dermatophyte reinfection from shoes to skin and vice-versa.

Description of the Organism-Specific Enzymes and Optional Active Ingredients

The fungal cell wall contains various structural components, especially chitin (a polymer of β-1,4-linked N-acetylglucosamine) and certain glucans (variously linked polymers of glucose), that are unique and essential to maintaining the integrity and viability of fungal cells, and that are not found in mammalian or bacterial cells. Fungal cell walls vary considerably in the proportion of chitin, glucan, mannan, and other carbohydrates that they contain. For example, yeast cell walls can contain as little as 1% chitin and are high in glucan and mannan content, whereas filamentous fungi such as dermatophytes can contain as much as 20% chitin by weight, with glucan and mannan contents being somewhat lower than in yeasts (Georgopapadakou, N., and Tkacz, J., 1995, Trends Microbiol. 3:98-104).

Enzymes that degrade these structural components of cell walls are known, with specific types hydrolyzing the polymers either within the polymer chain (endo-acting) or from either end of the polymer (exo-acting). Preferred enzymes are those that hydrolyze the polymers in an endo fashion, since these more rapidly break up the long polymer chains that maintain the structural integrity of the cell wall. Furthermore, such enzymes may act synergistically, that is, their effect in combination is more than the sum of their effects individually. Synergism is thought to be especially pronounced with chitinases and glucanases, since the respective polymers are important in most fungal cell walls. This invention takes advantage of rapid hydrolysis of fungal cell wall polymers by endo-acting enzymes, especially by chitinases and glucanases in combination, to degrade dermatophyte cell walls, and thereby inhibit or prevent dermatophyte growth, or both.

Without wishing to be bound by theory, it is thought that the use of enzyme-based antifungal treatments to control dermatophyte growth has several advantages over treatments with traditional chemical antimicrobial and antifungal agents alone (without enzymes). Since fungal cell wall polymer contents vary among fungi, and since the polymers being degraded are not present in bacterial or mammalian cells, enzyme-based treating compositions can be found that are very specific to the fungal species being treated. This specificity of antifungal enzyme treatments is thought to be important for the treatment of human skin because there is increasing evidence that disruption of native skin microbial flora by traditional, broad-spectrum antimicrobial treatments may be damaging in the long-term. It is also thought that depositing enzyme-based antifungal treatments can provide a prolonged effect whereby dermatophyte growth is stopped or retarded for a period of time. Enzyme-based treatments also may have environmental benefits, since, by their very nature as proteins, they are biodegradable and may have negligible non-specific toxicity. In addition, because antifungal enzymes act on cell wall polymers derived from a complex cellular process that involves many highly-evolved components, and do not act on single cellular targets, the probability of resistant fungal strains developing is likely smaller than chemical agents that act on single targets. Furthermore, the variety of naturally occurring antifungal enzymes is staggering, and modern molecular methods (such as directed evolution) provide the means to tailor the specificity and activity of these enzymes for maximal potency.

Organism-Specific Enzyme Classes

Typically, the classes of organism-specific enzymes used in compositions for treatment of dermatophytic fungi comprise chitinases, glucanases, and combinations thereof. The classes of organism-specific enzymes may be used either separately or in combinations of one or more organism-specific enzymes from each class. Compositions may optionally further comprise one or more additional organism-specific enzymes such as an organism-specific enzyme from the classes β-1,6-glucanases (E.C. No. 3.2.1.75), β-1,4-mannanases (E.C. No. 3.2.1.78), β-1,6-mannanases (E.C. No. 3.2.1.101), chitosanases (E.C. No. 3.2.1.132), endoglycosidases (E.C. No. 3.2.1.96), or combinations thereof.

Chitinases

Endochitinases (E.C. No. 3.2.1.14) represent a preferred type of chitinase enzyme for control of dermatophytic fungi. The cloning, expression, and purification of a large number of chitinase genes has been described in detail in the literature, and will be readily available to those skilled in the art without undue experimentation (see for example, U.S. Pat. No. 5,433,947; U.S. Pat. No. 4,751,081; and WO 94/24271). Furthermore, chitinase genes are widely available, and can be obtained from environmental isolates of bacteria, can be engineered for improved function from natural starting genes, or can be obtained from microbial culture collections or other sources. Examples of suitable chitinases for use in the control of dermatophytes include, but are not limited to, chitinases and their genes obtained from Trichoderma harzianum (including those derived from strain P1, American Type Culture Collection [ATCC] Accession No. 74058), Serratia marcescens, Aspergillus nidulans, Bacillus circulans, Bacillus licheniformis, Bacillus subtilis, Streptomyces griseus, and a wide variety of other bacteria and fungi. Chitinases are also commercially available. Partially purified chitinase preparations are available from Sigma Chemical Company and CalBiochem.

Preferred chitinases for use in this invention have pH optima for activity on dermatophyte cell walls such that the pH optimum is similar to the pH of the composition in which the chitinase is formulated or such that the pH optimum is similar to the pH of the substrate on which the chitinase will be applied (e.g., skin or shoes), or both.

Glucanases

Preferred types of glucanases include, but are not limited to, β-1,3-glucanases (E.C. No. 3.2.1.39), β-1,3-(1,4)-glucanases (E.C. No. 3.2.1.6; laminarases), lichenases (E.C. No. 3.2.1.73), and combinations thereof. More preferred glucanases include β-1,3-glucanases (E.C. No. 3.2.1.39). Optionally, β-1,6-glucanases (E.C. 3.2.1.75) can also be used in this invention.

The cloning, expression, and purification of large quantities of a large number of glucanase genes has been described in detail in the literature and is readily available to those skilled in the art without undue experimentation (see for example, U.S. Pat. No. 5,433,947; U.S. 4,751,081; and WO 94/24271). Furthermore, glucanase genes are widely available, and can be obtained from environmental isolates of bacteria, can be engineered for improved function from natural starting genes, or can be obtained from microbial culture collections or other sources. Examples of suitable sources of glucanases for use in the control of dermatophytes includes, but are not limited to, glucanases and their genes obtained from Arthrobacter luteus, Oerskovia xanthineolytica (including those derived from strain LLG109 DSM Accession No. 10297), Bacillus species (including Bacillus circulans), Streptomyces species, and a wide variety of other bacteria and fungi.

β-1,3-glucanases are also used in the processing of some foods and beverages, hence are readily available in commercial quantities. Suppliers include Novozymes A/S, DSM Gist, Danisco Ingredients, Biocatalysts Limited, Aventis Animal Nutrition, Diagnostic Chemicals Limited, K-I Chemical Industry Co. Ltd., Quest International Ireland Ltd., Rhodia Limited, and Takeda Vitamin & Food USA Inc.

Typically, glucanases for use in the invention disclosed have pH optima for activity on dermatophyte cell walls such that the pH optimum is similar to the pH of the composition in which the glucanase is formulated or such that the pH optimum is similar to the pH of the substrate on which the glucanase will be applied (e.g., skin or shoes), or both.

Optional Antifungal Agents

Without wishing to be bound by theory, it is thought that the antifungal effect of traditional antifungal agents (fungicides) can be boosted significantly if they are applied in conjunction with antifungal enzymes, such as the organism specific enzymes described above. Since the cell wall is a barrier to many of these fungicides, it is thought that degradation of the wall by antifungal enzymes allows better access of the fungicides to the cell membrane and cytosol, where they are active. In practical terms, this combination of enzymes with traditional antifungal agents means that much less antifungal agent can be used, resulting in cost savings, reduced toxicity, and hence possibility of application in compositions or contexts not previously practical.

Examples of antifungal agents suitable to gain one or more of the benefits described above when used in combination with antifungal enzymes include, but are not limited to, miconazole nitrate, terbinafine hydrochloride, clotrimazole, ketoconazole, flusilazole, tolnaftate, undecelynic acid, nystatin, amphotericin B, sorbic acid, sorbose, benzoic acid, propionic acid, methyl paraben, propyl paraben, captan, orthophenylphenol, nisin, natamycin, gramicidin, zinc pyrithione (ZPT), detergents, lytic peptides, membrane-affecting enzymes such as phospholipase B, and combinations thereof.

Compositions

This invention further relates to compositions comprising the organism specific enzyme described above. The exact amount of each organism-specific enzyme in the composition will depend on various factors including the method of use of the composition selected (e.g., topical or treatment, dilute in wash water or direct application to a substrate, etc.). However, each organism-specific enzyme (chitinase, glucanase, and any optional enzyme described) is typically present in the compositions described herein to a final concentration of about 0.1 parts per million (ppm) to about 1000 ppm. Typically, the ratio of chitinase to glucanase is about 0.01:1 to about 100:1.

Antifungal agents are typically added to the compositions described herein in amounts of 0 to about 10,000 ppm, preferably about 0.1 ppm to about 10,000 ppm. Typically, the ratio of agent to total organism-specific enzyme amount in the composition is 0 to about 1,000:1, preferably about 0.001:1 to about 1,000:1.

The composition may further comprise enzyme stabilizing agents including polyols such as 1,2-propanediol, glycerol, polyethylene glycol, or combinations thereof; salts; preservatives; and protease inhibitors or stabilizing systems; or combinations thereof.

Treating Compositions

This invention further relates to treating compositions that can be used to treat various articles such as shoes. The treating compositions comprise the organism-specific enzyme described above, at amounts described above.

The treating compositions can be in the form of a solid (powder, granules, bars, tablets), liquid, paste, gel, spray, aerosol, stick, foam, and combinations thereof. Preferred treating compositions can be easily spread or applied to all targeted or intended shoe surfaces by the user. Typically, treating compositions are formulated such the organism-specific enzymes and other actives are sufficiently stable that an adequate product shelf life is obtained while maintaining product efficacy.

In one embodiment of the invention, the organism-specific enzyme described above can be incorporated into a product for treating shoes such as that disclosed in WO 01/30955, which is hereby incorporated by reference. The product can be a used for cleaning or conditioning shoes, or both. The product can include a composition comprising an organism-specific enzyme described above and a benefit agent. The benefit agent can be a cleaning system benefit agent, a conditioning system benefit agent, a disinfecting system benefit agent, a conventional benefit agent, combinations thereof, and others.

Suitable cleaning composition benefit agents are disclosed at pp. 15-52 of WO 01/30955. Examples include calcium/magnesium removal agents, surfactants, dispersants/anti-redeposition agents, and combinations thereof. The cleaning compositions can have various forms including gels, pastes, liquids, granules, and others.

Suitable conditioning composition benefit agents are disclosed at pp. 54-59 of WO 01/30955. Suitable conditioning composition benefit agents include conditioning agents, perfumes, perfume delivery systems, anti-microbials and anti-fungals other than the organism-specific enzymes of this invention, malodor reduction technologies, cleaning technologies, combinations thereof, and others. The conditioning compositions can have various forms including gels, pastes, liquids, granules, and others.

Suitable disinfecting composition benefit agents are disclosed at pp. 61-63 of WO 01/30955. Suitable disinfecting composition benefit agents include surface active agents, bleaches, antimicrobial amphoteric compounds, organic and inorganic acids and their esters and salts, aromatic diamidines, biguanides, aldehydes, alcohols and phenols, nitrogen containing compounds, chelating agents, perfumes and essential oils, combinations thereof, and others.

One or more conventional benefit agents/adjuncts may be optionally be added to the compositions described above, in addition to, or instead of, the benefit agents described above. Suitable conventional benefit agents/adjuncts are disclosed at pp. 76-94 of WO 01/30955. Suitable conventional benefit agent/adjuncts include chelating agents, spreading agents, brighteners, suds suppressors, dye transfer inhibiting agents, preservatives, bleaching systems, bleaching agents, enzymes, solvents, buffers, combinations thereof, and others.

One or more other benefit agents may be optionally be added to the compositions described above, in addition to, or instead of, the benefit agents described above. Such benefit agents are disclosed at pp. 63-75 of WO 01/30955. Such benefit agents include release agents, protease enzymes (that produce a cleaning, stain removal, soil removal, whitening, deodorizing, or freshness improving effect on substrates), enzyme stabilizers, odor control agents, perfumes, sustained perfume release agents, film forming polymers, combinations thereof, and others.

In one embodiment of this invention, a treating composition for treating a shoe comprises an organism-specific enzyme for treating microbes in the shoe when the treating composition is applied directly or indirectly to the shoe prior to and/or during and/or after washing the shoe with or in an aqueous medium, wherein said treating composition is formulated so that any damage as a result of washing the shoe with or in an aqueous medium containing the treating composition is reduced compared to washing the shoe with or in an aqueous medium free of the treating composition. The treating composition may optionally further comprise a benefit agent selected from the group consisting of: cleaning agents, conditioning agents, disinfecting agents, antibacterial agents, antimicrobial agents, antifungal agents, odor control agents, waterproofing agents, soil release agents, brightening agents, alkaline pH modifiers, perfume, and mixtures thereof.

In an alternative embodiment of the invention a treating composition comprises:

a) an organism-specific enzyme,

b) a cleaning agent, and

c) a conditioning agent,

wherein cleaning benefits and/or conditioning benefits are imparted to the one or more shoes when the treating composition is applied to the one or more shoes prior to and/or during and/or after washing the one or more shoes.

In an alternative embodiment of the invention, a treating composition comprises:

a) a cleaning composition comprising a cleaning agent capable of being applied in a manner such that the cleaning agent contacts an exterior surface of the shoe; and

b) a conditioning composition physically and/or chemically separated from the cleaning composition of a) wherein the conditioning composition comprises a conditioning agent capable of being applied in a manner such that the conditioning agent contacts an interior surface of the shoe;

such that the cleaning composition and/or conditioning composition imparts cleaning benefits and/or conditioning benefits to the shoe when the cleaning composition and/or conditioning composition are applied to the shoe prior to and/or during and/or after washing the shoe.

Methods of Use of the Treating Compositions

This invention further relates to methods for treating shoes. The method comprises comprising contacting a shoe with an organism-specific enzyme, as described above. Typically, a treating composition comprising the organism-specific enzyme is used for contacting the shoe. The treating composition can be applied to an interior surface of the shoe, an exterior surface of the shoe, or both.

The shoe can be contacted prior to and/or during and/or after washing the shoes with or in an aqueous medium. In one embodiment of the invention, the treating composition is applied in the wash cycle of a washing machine. In one embodiment of the invention, the shoe can be placed in a containment bag, and the bag can be placed into a wash solution. In this embodiment, the containment bag may contain a treating composition, the wash solution may comprise the treating composition, or the treating composition can be in both the containment bag and the wash solution. Suitable methods and containment bags for treating shoes are disclosed in WO 01/30955 at pp. 94-105. In a preferred embodiment of the invention, the treating composition is directly applied to the shoes. This offers the benefit of ensuring that a higher concentration of organism-specific enzyme contacts the shoe (e.g., higher than if the treating composition is diluted in wash water) or allows a smaller amount of organism-specific enzyme to be used in the treating composition (e.g., smaller than if the treating composition is diluted in wash water).

Various types of shoes can be treated using the compositions and methods described above. For example, a shoe to be treated may comprise canvas, nylon, synthetic leather, natural leather, or combinations thereof. In one embodiment of the invention, the shoe is an athletic shoe being treated for athlete's foot.

In an alternative embodiment of the invention, the compositions described herein can be used in an aqueous environment. For example, the compositions may be used in the wash cycle of a washing machine, the rinse cycle of a washing machine, and combinations thereof. Alternatively, the organism-specific enzyme described above can be formulated in a commercially available detergent, such as liquid Tide (commercially available from the Procter & Gamble Company of Cincinnati, Ohio) or a commercially available fabric softener, such as Downy (commercially available from the Procter & Gamble Company of Cincinnati, Ohio). The organism-specific enzyme is added in quantities sufficient to achieve the benefits described above upon dilution or with direct application to the infected substrate (e.g., socks, shoes, or other clothing).

Topical Compositions

This invention further relates to topical compositions for application to skin. Topical compositions comprise: component (A), the organism-specific enzyme described above, and component (B) a topical carrier. The topical carrier preferably aids penetration of component (A) into the skin. Topical compositions preferably further comprise (C) an optional ingredient, such as an additional organism-specific enzyme, an antifungal agent, or combinations thereof, described above.

Component (B) the topical carrier may comprise a single ingredient or a combination of two or more ingredients. Preferred topical carriers comprise one or more ingredients selected from the group consisting of water, alcohols, aloe vera gel, allantoin, glycerin, vitamin A and E oils, mineral oil, propylene glycol, polypropylene glycol-2 myristyl propionate, dimethyl isosorbide, combinations thereof, and the like.

The topical carrier may comprise one or more ingredients selected from the group consisting of a) emollients, b) propellants, c) solvents, d) humectants, e) thickeners, f) powders, g) fragrances, and h) waxes in addition to, or instead of, the preferred topical carrier ingredients listed above. One skilled in the art would be able to optimize topical carrier ingredients for the topical compositions without undue experimentation.

Ingredient a) is an emollient. The amount of ingredient a) in the topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petrolatum, mineral oil, butyl myristate, isostearic acid, palmitic acid, isopropyl linoleate, lauryl lactate, myristyl lactate, decyl oleate, myristyl myristate, polydimethylsiloxane, and combinations thereof.

Ingredient b) is a propellant. The amount of ingredient b) in the topical composition is typically about 5 to about 95%. Suitable propellants include propane, butane, isobutane, dimethyl ether, carbon dioxide, nitrous oxide, nitrogen, and combinations thereof.

Ingredient c) is a solvent. The amount of ingredient c) in the topical composition is typically about 5 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof.

Ingredient d) is a humectant. The amount of ingredient d) in the topical composition is typically about 5 to about 95%. Suitable humectants include glycerin, sorbitol, sodium 2-pyrrolidone-5-carboxylate, soluble collagen, dibutyl phthalate, gelatin, and combinations thereof.

Ingredient e) is a thickener. The amount of ingredient e) in the topical composition is typically 0 to about 95%.

Ingredient f) is a powder. The amount of ingredient f) in the topical composition is typically 0 to about 95%. Suitable powders include chalk, talc, fullers earth, kaolin, starch, gums, colloidal silicon dioxide, sodium polyacrylate, tetraalkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

Ingredient g) is a fragrance. The amount of ingredient g) in the topical composition is typically about 0.001 to about 0.5%, preferably about 0.001 to about 0.1%.

Ingredient h) is a wax. Waxes useful in this invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point between 40 and 100° C. The amount of ingredient h) in the topical composition is typically about 1 to about 99%.

In an alternative embodiment of the invention, component (A) may also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines. A preferred composition for topical delivery of the present compounds uses liposomes as described in Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Wallach and Philippot, “New Type of Lipid Vesicle: Novasome®”, Liposome Technology, Vol. 1, pp. 141-156 (1993); U.S. Pat. No. 4,911,928, and U.S. Pat. No. 5,834,014.

The exact amounts of each component in the topical composition depend on various factors. Including the specific enzyme selected for component (A) and the mode by which the composition will be administered. However, the amount of component (A) typically added to the topical composition is about 0.1 to about 99%, preferably about 1 to about 10%. The topical composition preferably further comprises 0 to about 99% component (C), more preferably 0 to abut 10%, and a sufficient amount of component (B) such that the amounts of components (A), (B), and (C), combined equal 100%. The amount of (B) the carrier employed in conjunction with component (A) is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Topical compositions that can be applied locally to the skin may be in any form including solutions, oils, creams, ointments, gels, lotions, shampoos, leave-on and rinse-out hair conditioners, milks, cleansers, moisturizers, sprays, skin patches, and the like.

Methods of Use of the Topical Compositions

This invention further relates to methods of use of the topical compositions. The topical compositions are particularly well suited for treating a subject suffering from a microbial infection on skin. The composition is applied to the affected area at least once per day.

In a preferred embodiment, a method for treating fungal infections comprises applying to skin an effective amount of an organism-specific enzyme comprising an endochitinase and a glucanase. In this embodiment, the glucanase is preferably selected from the group consisting of a β-1,3-glucanase, a β-1,3-(1,4)-glucanase, lichenase, and combinations thereof. The organism-specific enzyme preferably further comprises one or more additional organism-specific enzymes selected from the group consisting of a β-1,6-glucanase, a β-1,4-mannanase, a β-1,6-mannanase, chitosanase, and an endoglycosidase.

Kits

Component (A) may be included in kits comprising component (A), a treating composition described above, a topical composition described above, or combinations thereof; and information, instructions, or both that use of the kit will provide treatment for microbial infections (particularly in humans). The information and instructions may be in the form of words, pictures, or both, and the like. In addition or in the alternative, the kit may comprise component (A), a treating composition described above, a topical composition described above, or combinations thereof; and information, instructions, or both, regarding methods of administration of component (A) or the compositions, preferably with the benefit of treating microbial infections in mammals.

Methods of Use

This invention further relates to a method for treating a microbial infection comprising:

a) treating skin with a first organism-specific enzyme, and

b) treating a substrate that, directly or indirectly, covers the skin with a second organism-specific enzyme, wherein the first and second organism-specific enzymes may be the same or different.

This method can be used to treat various types of microbial infections. One skilled in the art would be able to select appropriate first and second organism-specific enzymes for use in the method, depending on the type of microbial infection to be treated. In a preferred embodiment of the invention, the method can be used to treat infections in the skin of a foot, such as tinea pedis. In this embodiment, the skin is on a foot and the substrate is selected from the group consisting of a sock, a shoe, and combinations thereof.

In a preferred embodiment of the invention, the first and second organism-specific enzymes are each independently selected from the group consisting of a chitinase, a glucanase, and combinations thereof. Typically, a topical composition comprising the first organism-specific enzyme and a topical carrier is used in step a). Typically, a treating composition comprising the second organism-specific enzyme and a benefit agent is used in step b).

### EXAMPLES

Abbreviations

| | |
|---|---|
| μg | micrograms |
| ° C. | degrees Celsius |
| cfu | colony forming units |
| cm | centimeters |
| ml | milliliters |
| mM | millimolar |
| ppm | parts per million |
| rpm | revolutions per minute |

### Example 1

Efficacy of Organism Specific Enzymes Against Dermatophytes

Purified arthrospore suspensions of T. mentagrophytes or T. rubrum (prepared as per L. R. Wright et al., 1983, J. Antimicrob. Chemotherapy 12:317-327), diluted to a concentration of 1×10⁶ cfu/ml in Emmon's modification of Sabouraud Dextrose Broth (ESD—R. Atlas, 1997, Handbook of microbiological media, CRC Press Inc., Boca Raton, Fla.), are incubated for 6 hours at 26° C. or 30° C., respectively, to pre-germinate spores. Enzyme preparations of chitinase (Sigma Cat. No. C-1525) and β1-1,3-glucanase (Sigma Cat. No. L-2524) are diluted into 3 ml volumes of ESD in capped glass tubes, to final concentrations of each enzyme ranging from 0 to 100 ppm. Clotrimazole controls are also prepared, to a final concentration of 20 μ/ml per tube.

Each tube is then inoculated with 10 μl (1×10⁴ cfu) of either the T. mentagrophytes or T. rubrum pre-germinated arthrospore suspension, followed by incubation on a shaker at 200 rpm, at 26° C. or 30° C., respectively, for a total of 41 hours. Relative growth in the tubes is scored at 18 and 41 hours, as shown in Table 1.

TABLE 1

| [enzymes], | T. mentagrophytes* | | T. rubrum* | |
|---|---|---|---|---|
| ppm | 18 hours | 41 hours | 18 hours | 41 hours |
| 0 | ++ | +++ | ++ | +++ |
| 1 | − | +++ | + | +++ |
| 10 | − | +++ | − | +++ |
| 50 | − | + | − | ++ |
| 100 | − | − | − | − |
| 100 ppm | + | ++ | − | + |
| chitinase alone | | | | |
| 100 ppm β- | + | ++ | + | ++ |
| 1,3-glucanase | | | | |
| alone | | | | |
| clotrimazole | − | − | − | − |

*Growth is scored qualitatively, using a relative scale from - (no growth) to +++ (maximal growth).

The results indicate combining chitinase with β-1,3-glucanase, and that 100 ppm of each organism-specific enzyme preparation (in combination) is sufficient to inhibit growth of either dermatophyte species for at least 41 hours, using the conditions specified.

### Example 2

Shoe Conditioner Compositions

Shoe conditioning agent-containing treating compositions useful in the laundering of shoes as described in WO 01/30955 are formulated as follows:

| | Example A | Example B | Example C | Example D | Example E |
|---|---|---|---|---|---|
| Component | Weight % (Active Weight %) | | | | |
| Conditioning Agent¹ | 33(12) | 40(15) | 33(12) | 33(12) | 33(12) |
| Substantive | 0.3 | 0.3 | 0.3 | 0.3 | 0.3 |
| Perfume | | | | | |
| Anti-fungal enzyme | 200 ppm | 200 ppm | 200 ppm | 200 ppm | 200 ppm |
| system (active enzyme)² | | | | | |
| Antimicrobial | 0-3 | 0-3 | 0-3 | 0-3 | 0-3 |
| preservative³ | | | | | |
| Nonionic Surfactant⁴ | 0 | 0 | 1.0 | 1.0 | 1.0 |
| Odor Control Agent⁵ | 0 | 2.0(1.0) | 0 | 0 | 0 |
| Propylene glycol | 0 | 0 | 0 | 0 | 4.0 |
| Water | balance | balance | balance | balance | balance |
| pH adjusted by any | 4-6 | 4-6 | 4-6 | 4-6 | 4-6 |
| suitable means | | | | | |

¹A suitable conditioning agent is commercially available under the tradename LUBRITAN AS from Rohm and Haas Company.
²100 ppm each of chitinase and β-1,3-glucanase.
³A suitable antimicrobial preservative may be selected from the group of: methyl parabens, ethyl parabens, Benzoic acid and its salts, Sorbic acid and its salts, Polyhexamethylenebiguanide, Chlorhexidine, orthophenylphenol, and combinations thereof.
⁴A suitable nonionic surfactant is commercially available under the tradename NEODOL 23-6.5 from Shell Chemical Company.
⁵A suitable odor control agent is β-cyclodextrin.

### Example 3

Treating Composition

A cleaning agent-containing treating composition useful in the laundering of shoes as described in WO 01/30955 is formulated as follows:

| | Formula % |
|---|---|
| Sodium Polyacrylate¹ | 39.35 |
| Nonionic Surfactant² | 11.67 |
| Antimicrobial preservative³ | 0-3% |
| Anti-fungal enzyme system (active | 200 ppm |
| enzyme)⁴ | |
| Silicone suds suppresser | 0.6 |
| Perfume | 0.25 |
| Water | balance |
| Minors (dyes, etc.) | 0.13 |
| Total | 100.00 |

¹A suitable sodium polyacrylate is commercially available under the tradename ACUSOL 445N (45% active) from Rohm and Haas Company.
²A suitable nonionic surfactant is commercially available under the tradename NEODOL 23-9 from Shell Chemical Company.
³A suitable antimicrobial preservative may be selected from the group of: methyl parabens, ethyl parabens, Benzoic acid and its salts, Sorbic acid and its salts, Polyhexamethylenebiguanide, Chlorhexidine, orthophenylphenol, and combinations thereof
⁴100 ppm each of chitinase and β-1,3-glucanase.

### Example 4

Treating Composition

A treating composition useful in the laundering of shoes as described in WO 01/30955 is formulated as follows:

| | |
|---|---|
| Acrylic Acid/Maleic Acid Copolymer (1) | 29.8% |
| Nonionic Surfactant (2) | 12% |
| Glycerin | 2% |
| 2,2,4-Trimethyl-1,3-Pentanediol | 1% |
| Silicone Suds Suppressor | 0.2% |
| Anti-fungal enzyme system (active enzyme) (3) | 200 ppm |
| Antimicrobial preservative (4) | 0-3% |
| Thickening Agent (5) | 0.2% |
| Substantive Perfume | 0.2% |
| Minors (dye, perfume, preservative) | 0.1% |
| Water | balance |

(1) A suitable Acrylic Acid/Maleic Acid copolymer is commercially available under the tradename SOKALAN CP-5 (40% active) from BASF.
(2) Commercially available under the tradename NEODOL 23-9 from Shell Chemical Co.
(3) 100 ppm each of chitinase and β-1,3-glucanase.
(4) A suitable antimicrobial preservative may be selected from the group of: methyl parabens, ethyl parabens, Benzoic acid and its salts, Sorbic acid and its salts, Polyhexamethylenebiguanide, Chlorhexidine, orthophenylphenol, and combinations thereof.
(5) Trihydroxystearin.

### Example 5

Treating Composition

A cleaning agent and conditioning agent-containing treating composition (2-in-1) is formulated as follows:

| | A | B |
|---|---|---|
| Acrylic acid/Maleic acid Copolymer¹ | 30.9% | 30.9% |
| Nonionic Surfactant² | 13.6% | 13.6% |
| Conditioning Agent³ | 2.5% | 2.5% |
| Anti-fungal enzyme system (active enzyme)⁴ | 200 ppm | 200 ppm |
| Antimicrobial preservative⁵ | 0-3% | 0-3% |
| Silwet L-7500⁶ | 1.8% | 1.8% |
| Substantive Perfume | — | 0.5% |
| Water | balance | balance |
| | 100.0% | 100.0% |

¹A suitable acrylic acid/maleic acid copolymer is commercially available under the tradename SOKALAN CP-5 (40% active) from BASF.
²A suitable nonionic surfactant is commercially available under the tradename NEODOL 23-9 from Shell Chemical Company.
³A suitable conditioning agent is commercially available under the tradename LUBRITAN AS from Rohm and Haas Company.
⁴100 ppm each of chitinase and β-1,3-glucanase.
⁵A suitable antimicrobial preservative may be selected from the group of: methyl parabens, ethyl parabens, Benzoic acid and its salts, Sorbic acid and its salts, Polyhexamethylenebiguanide, Chlorhexidine, orthophenylphenol, and combinations thereof.
⁶Silwet L-7500 is available from OSI Specialties.

### Example 6

Antifungal Treating Composition

A treating composition especially useful as an anti-fungal compositions is formulated as follows:

| Distilled H₂O | balance |
|---|---|
| Anti-fungal enzyme | 200 ppm |
| system (active enzyme)¹ | |
| Antimicrobial | 0-3% |
| preservative² | |
| Isopropanol | 2% |
| Perfume³ | 0.5% |
| Malic acid | To adjust pH |
| final pH | 4.5 |

¹100 ppm each of chitinase and β-1,3-glucanase.
²A suitable antimicrobial preservative may be selected from the group of: methyl parabens, ethyl parabens, Benzoic acid and its salts, Sorbic acid and its salts, Polyhexamethylenebiguanide, Chlorhexidine, orthophenylphenol, and combinations thereof.
³Substantive perfume containing at least about 30% of substantive perfume ingredients.

### Example 7

Efficacy of the Treating Compositions

(a). Purified arthrospore suspensions of T. mentagrophytes (prepared as specified in Example 1), diluted to a concentration of 1×10⁷ cfu/ml in ESD, are incubated for 6 hours at 26° C. to pre-germinate spores. Enzyme preparations of chitinase (Sigma Cat. No. C-1525) and β-1,3-glucanase (Sigma Cat. No. L-2524) are diluted into 100 μl of volumes of 25 mM 2-[N-morpholino]ethanesulfonic acid (MES), pH 6.0, or into volumes of 10% or 20% of the product composition specified in Example 1, Composition A (diluted in water), to give final concentrations of each enzyme of 100 ppm. Pre-sterilized (autoclaved) 1.44 cm² pieces of shoe insole (AirFlow model, Quality Brands, Grand Rapids, Mich.) are inoculated with 50 μl of the T. mentagrophytes pre-germinated arthrospore suspension, are allowed to incubate at room temperature (about 25° C.) for 5 minutes, then are treated with either the 100 μl of enzyme dilutions described above (enzyme treated samples), or treated with 100 μl volumes of 25 mM MES (pH 6.0), 10%, or 20% of the product composition (diluted in water) that did not contain any enzyme (control samples). Treated insole pieces are incubated at room temperature for 30 minutes, in wells of a polystyrene 24-well microplate, then are removed and pressed inoculum side down onto the surface of Dermatophyte Test Medium agar plates (DTM—R. Atlas, 1997, Handbook of microbiological media, CRC Press Inc., Boca Raton, Fla.), that are modified to omit the phenol red dye and cycloheximide and are adjusted to pH 6.0. After removal of the pieces from the agar surface, plates are incubated at 26° C. for about 5 days, and are then imaged with a digital camera and light box.

Images are analyzed for the area of the insole impression covered by fungal growth, and results are expressed as a percentage reduction of fungal growth due to the effects of enzyme treatment, relative to the control sample treatments that contained no enzyme, as shown in Table 2.

TABLE 2

Buffer or Product Composition Dilution

| | 25 mM MES | 10% Product | 20% Product |
|---|---|---|---|
| | (pH 6.0) | Composition | Composition |
| % reductions of | 88% | 78% | 97% |
| fungal growth, | | | |
| relative to control | | | |
| samples | | | |

The data indicate that the enzyme treatment significantly reduces the growth of T. mentagrophytes on shoe insoles, when treatment is administered in the presence of the product composition used.

(b). A similar experiment is conducted in duplicate, where larger insoles (˜6.5 cm²) are used, where the volumes of treatments and inocula are scaled appropriately, and where both T. mentagrophytes and T. rubrum are tested, but otherwise using the same method as described in (a) above, with the 20% product composition dilution and 100 ppm of each enzyme. The results, shown in Table 3, are expressed in terms of percentage reduction of fungal growth, in the same way as described for Table 2.

TABLE 3

Percentage Reduction of Fungal Growth

| | T. rubrum | T. mentagrophytes |
|---|---|---|
| Experiment 1 | 73% | 99% |
| Experiment 2 | 87% | 76% |

These results confirm the efficacy of the dual enzyme treatment in the presence of the product composition, for both dermatophyte species.

### Example 8

Shoe Treatment Kit

A shoe treatment kit is prepared by combining the conditioning system from Example 2, a shoe cleaning gel from Example 3 or Example 4, and a shoe bag as described in WO 01/30955 and used with the above cleaning and conditioning systems and all used as described in WO 01/30955.

What is claimed is:

1. A method for treating a microbial infection comprising:
a) treating skin with a first organism-specific enzyme, and
b) treating a substrate that, directly or indirectly, covers the skin with a second organism-specific enzyme, wherein the first and second organism-specific enzymes may be the same or different.

2. The method of claim 1, wherein the skin is on a foot and the substrate is selected from the group consisting of a sock, a shoe, and combinations thereof.

3. The method of claim 1, wherein the first and second organism-specific enzymes are each independently selected from the group consisting of a chitinase, a glucanase, a β-1,6-glucanase, β-1,4-mannanases, β-1,6-mannanases, chitosanases, endoglycosidases, and combinations thereof

4. The method of claim 3, wherein the first and second organism-specific enzymes are each independently selected from the group consisting of a chitinase, a glucanase, and combinations thereof.

5. The method of claim 1, wherein a topical composition comprises the first organism-specific enzyme and a topical carrier.

6. The method of claim 1, wherein a treating composition comprises the second organism-specific enzyme and a benefit agent.

7. A method of treating a shoe comprising contacting the shoe with an organism-specific enzyme.

8. The method of claim 7, wherein the organism-specific enzyme is selected from the group consisting of chitinases, glucanases, and combinations thereof.

9. The method of claim 7, wherein a treating composition comprising the organism-specific enzyme is used for contacting the shoe.

10. The method of claim 9, wherein the shoe is contacted prior to and/or during and/or after washing the shoes with or in an aqueous medium.

11. The method of claim 10, wherein the treating composition is applied in the wash cycle of a washing machine.

12. The method of claim 9, further comprising placing the shoe in a containment bag, and placing the bag into a wash solution.

13. The method of claim 12, wherein:
the containment bag contains a treating composition;
the wash solution comprises a treating composition; or
the treating composition is in both the containment bag and the wash solution.

14. The method of claim 9, wherein the treating composition is applied to one or more interior surfaces of the shoe; to one or more exterior surfaces of the shoe; or both.

15. The method of claim 7, wherein the shoe comprises canvas, nylon, synthetic leather, natural leather, or combinations thereof.

16. A method for treating fungal infections comprising: applying to skin an effective amount of an organism-specific enzyme comprising an endochitinase and a glucanase.

17. The method of claim 16, wherein the glucanase is selected from the group consisting of a β-1,3-glucanase, a β-1,3-(1,4)-glucanase, lichenase, and combinations thereof.

18. The method of claim 16, wherein the organism-specific enzyme further comprises one or more enzymes selected from the group consisting of a β-1,6-glucanase, a β-1,4-mannanase, a β-1,6-mannanase, a chitosanase, and an endoglycosidase.

19. A treating composition for treating a shoe comprising an organism-specific enzyme for treating microbes in the shoe when the treating composition is applied directly or indirectly to the shoe prior to and/or during and/or after washing the shoe with or in an aqueous medium, wherein said treating composition is formulated so that any damage as a result of washing the shoe with or in an aqueous medium containing the treating composition is reduced compared to washing the shoe with or in an aqueous medium free of the treating composition.

20. The treating composition of claim 19, wherein the treating composition further comprises a benefit agent selected from the group consisting of: cleaning agents, conditioning agents, disinfecting agents, antibacterial agents, antimicrobial agents, antifungal agents, odor control agents, waterproofing agents, soil release agents, brightening agents, alkaline pH modifiers, perfume, and mixtures thereof.

21. The treating composition of claim 19, wherein the treating composition comprises:
a) an organism-specific enzyme,
b) a cleaning agent, and
c) a conditioning agent,
wherein cleaning benefits and/or conditioning benefits are imparted to the one or more shoes when the treating composition is applied to the one or more shoes prior to and/or during and/or after washing the one or more shoes.

22. The composition of claim 19, wherein the treating composition comprises:
a) a cleaning composition comprising a cleaning agent capable of being applied in a manner such that the cleaning agent contacts an exterior surface of the shoe; and
b) a conditioning composition physically and/or chemically separated from the cleaning composition of a) wherein the conditioning composition comprises a conditioning agent capable of being applied in a manner such that the conditioning agent contacts an interior surface of the shoe;
such that the cleaning composition and/or conditioning composition imparts cleaning benefits and/or conditioning benefits to the shoe when the cleaning composition and/or conditioning composition are applied to the shoe prior to and/or during and/or after washing the shoe.

23. A treating composition comprising:
A) an organism-specific enzyme, and
B) a benefit agent,
wherein the treating composition is used for treating a shoe.

24. A topical composition comprising:
(A) an organism-specific enzyme comprising an endochitinase and a glucanase, and
(B) a topical carrier.

25. The composition of claim 24, wherein the glucanase is selected from the group consisting of a β-1,3-glucanase, a β-1,3-(1,4)-glucanase, lichenase, and combinations thereof.

26. The composition of claim 24, wherein the organism-specific enzyme further comprises one or more enzymes selected from the group consisting of a β-1,6-glucanase, a β1,4-mannanase, a β-1,6-mannanase, a chitosanase, and an endoglycosidase.

27. The composition of claim 24, wherein the topical carrier comprises an ingredient selected from the group consisting of a) emollients, b) propellants, c) solvents, d) humectants, e) thickeners, f) powders, g) fragrances, h) waxes and combinations thereof.

28. The composition of claim 24, further comprising an antifungal agent.

29. A kit for treating a microbial infection comprising:
a) a treating composition;
b) a topical composition; and
c) optionally, information, instructions, or both on use of the kit to treat the microbial infection.
